(19) United States
(12) Patent Application Publication
(10) Pub. No.: US 2026/0174478 A1
(43) Pub. Date: Jun. 25, 2026

(54) CONNECTOR DEVICES, SYSTEMS AND METHODS

(71) Applicants: Oluwatodimu Richard RAJI, San Francisco, CA (US); Dimitriy KONDRASHOV, Sherman Oaks, CA (US); Jeremi Michael LEASURE, Carlsbad, CA (US)

(72) Inventors: Oluwatodimu Richard RAJI, San Francisco, CA (US); Dimitriy KONDRASHOV, Sherman Oaks, CA (US); Jeremi Michael LEASURE, Carlsbad, CA (US)

(21) Appl. No.: 19/427,598

(22) Filed: Dec. 19, 2025

Related U.S. Application Data
(60) Provisional application No. 63/736,475, filed on Dec. 19, 2024.

(51) Int. Cl. A61B 17/70; A61B 17/00; A61B 17/56

(52) U.S. Cl. CPC A61B 17/7086; A61B 17/7032; A61B 2017/00477; A61B 2017/564

(57) ABSTRACT

Provided are connector devices, systems, kits and methods that include a main body defining an opening configured for insertion of a primary rod, rail or plate therein, and a tulip over the main body, the tulip defining an opening configured for insertion of a secondary rod, rail or plate therein, the tulip being inserted into a top opening in the main body and attached to the main body directly above the main body, and a retention cap for maintaining the secondary rod, rail or plate at a desired position within the tulip. The main body and the tulip are aligned such that the openings are directly over each other in a second direction, thereby allowing a portion of the primary rod, rail or plate and a portion of the secondary rod, rail or plate to be alignable directly over one another in the second direction.

### CROSS REFERENCE TO RELATED APPLICATION

This application claims priority to and the benefit of U.S. Provisional Application 63/736,475 filed on Dec. 19, 2024, in the U.S. Patent and Trademark Office, the entire contents of which are incorporated herein by reference.

### FIELD

The present application relates generally to rod, rail or plate connector devices and systems that may be used in spinal surgery and methods of treating a patient using the present connector devices, systems and methods. Non-limiting example embodiments include rod, rail or plate connector devices, systems and methods for the minimally invasive, percutaneous insertion and placement of rod, rail or plate connectors within spinal rod, rail or plate fixation constructs.

### BACKGROUND

The evolution of minimally invasive surgical (MIS) techniques has significantly impacted the field of spinal surgery, aiming to reduce operative trauma and enhance patient recovery. Advantages of minimally invasive spine surgery including the use of percutaneous placement of pedicle screws in spine surgery include reduced surgical trauma, accelerated patient recovery, and a diminished incidence of postoperative complications such as post-op infection. These benefits represent a significant advancement in surgical practices and patient care outcomes.

Indications for the placement of spinal rods (or other connecting devices) can include:
backing up a long construct after multilevel Interbody Fusion surgeries, treatment of spinal fractures with or without neurological deficits, fixation of unstable 3-column Chance fractures, and fixation of unstable injuries in, ankylosed spine in patients with ankylosing spondylitis, diffuse idiopathic skeletal hyperostosis or ankylosis due to advanced degenerative disc disease.

A limitation of percutaneous fixation is the inability to place a connector device, such as a cross-link or a domino device, which connects or links e.g., two rods together. Alternatively, the present devices may link plates or rails or other suitable connection devices together.

Usage of connectors is widespread in open cervical and thoracolumbar fusion, and is especially useful for long constructs and compromised bone due to osteopenia or osteoporosis. Placing a parallel connector/domino creates a dual or multiple rod construct and decreases the chance of rod breakage by increasing rigidity, and placing a perpendicular/cross connector creates a fixed angle construct and decreases the chance of pullout of the screws-especially at the cephalad and caudal ends of the long construct. The inability to place a connector in a percutaneous construct is a significant limitation, as it can lead to construct failure due to screw pullout, and lessened rigidity of the construct, especially in highly unstable spine fractures.

### SUMMARY

The present application addresses the problems associated with conventional devices and methods and provides improvements on these devices.

Provided herein are connector devices and systems, including a main body defining a main body opening configured for insertion of a primary rod, rail or plate therein, a tulip defining a tulip opening configured for insertion of a secondary rod, rail or plate therein, the tulip being inserted into a top opening in the main body and attached to the main body above the main body, and a retention cap configured to maintain the secondary rod, rail or plate at a desired position therein within the tulip; in which the main body and the tulip are aligned in a first direction such that the main body opening and the tulip opening are directly over each other in a second direction that intersects the first direction, thereby allowing a portion of the primary rod, rail or plate and a portion of the secondary rod, rail or plate to be alignable directly over one another in the second direction.

Also provided herein are kits that include a main body and a tulip as set forth herein. Kits may also include a cannula, which may or may not be detachable, or one or more tabs configured to form a cannula upon attachment to the tulip. Kits may also include at least one additional component including instructions for use of the present systems, a primary rod, rail or plate and/or a secondary or further rod, rail or plate, a locking screw, a retention cap, or a tool for assembly or insertion of the rod connector system or a component thereof.

Further provided are methods of connecting multiple rods, rails or plates such as spinal rods, that include attaching a main body to a primary rod, rail or plate, attaching a tulip to the main body, attaching a tulip to the main body, placing a secondary rod, rail or plate into an opening of the tulip, adjusting the tulip or the secondary rod, rail or plate, such that the secondary rod, rail or plate has a desired angle with respect to the primary rail or plate, and driving a retention cap into the tulip to lock the secondary rod, plate or rail at a desired position, in which a portion of the primary rod, rail or plate within the main body is directly aligned in a vertical direction with a portion of the secondary rod, rail or plate within the tulip.

### BRIEF DESCRIPTION OF THE DRAWINGS

Non-limiting embodiments of the present application are demonstrated by the illustrative figures:

FIGS. 1A, 1B and 1C depict an embodiment of a main body in accordance with non-limiting examples, from side, bottom and top views, respectively. FIGS. 1A and 1B depict the main body having a primary rod therein.

FIGS. 2A and 2B depicts further embodiments of a main body having a bottom portion of a tulip engaged therewith in accordance with non-limiting examples. FIG. 2A is a cross-sectional view of the main body with a bottom portion of a tulip engaged therein.

FIGS. 3A and 3B depict an embodiment of a tulip in accordance with non-limiting examples. FIG. 3A depicts side view of an example tulip. FIG. 3B depicts a side cross-sectional view of an example tulip and also shows a secondary rod in the tulip, with a retention cap over the secondary rod.

FIG. 4 depicts a tulip within a main body in accordance with non-limiting examples.

FIG. 5 depicts a device or system including a main body, a tulip, and a cannula in accordance with non-limiting examples. In the depicted embodiment, side openings of the main body and the tulip are aligned with one another rotationally such that rods inserted in the side openings would be parallel with respect to each other.

FIG. 6 depicts a device or system including a main body, a tulip, and a cannula in accordance with non-limiting examples. In the depicted embodiment, the tulip is in a rotated position with respect to the main body such that side openings of the main body and the tulip are not aligned with one another rotationally such that rods inserted in the side openings would not be parallel with respect to each other. For example, the holes may be positioned such that rods therein may be perpendicular with respect to each other.

### DETAILED DESCRIPTION

Embodiments of the present disclosure will be described more fully hereinafter with reference to the accompanying drawings, in which embodiments of the disclosure are shown.

Example embodiments provided herein include rod connector devices, systems, and methods for the minimally invasive, percutaneous insertion and placement of rod connectors within spinal rod fixation constructs. Connectors may include for example, inline fixed angle configurations, such as domino, diagonal, or cross-connector configurations.

While embodiments are described herein, it is to be understood that this disclosure is illustrative and exemplary and is made for the purposes of providing a full and enabling disclosure. The detailed disclosure herein of example embodiments is not intended, nor is to be construed, to be limiting. All illustrations of the figures are for the purpose of describing selected embodiments and are not intended to limit the scope of the present application.

Each term used herein refers to that which an ordinary artisan would understand such term to mean based on the contextual use of such term herein. To the extent the meaning of a term used herein—as understood by the ordinary artisan differs in any way from any particular dictionary definition of such term, the meaning of the term as understood by the ordinary artisan in the context of the present application, should prevail.

Items described in the singular herein may be provided in plural, as can be seen, for example, in the drawings. Thus, the description of a single item that is provided in plural should be understood to be applicable to the remaining plurality of items unless context indicates otherwise.

The terms “a” or “an”, as used herein, are defined as one or more than one unless the contextual use dictates otherwise. The term plurality, as used herein, is defined as two or more than two. The term another, as used herein, is defined as at least a second or more.

When used herein to join a list of items, “or” denotes “at least one” of the items, but does not exclude a plurality of items of the list.

Throughout the specification, when a component is described as “including” a particular element or group of elements (or any version of “include”), it is to be understood that the component is formed of only the element or the group of elements, or the element or group of elements may be combined with additional elements to form the component, unless the context indicates otherwise.

It will be understood that when an element is referred to as being “connected” (or any form of “connect”) or “on” another element, it can be directly connected to or on the other element or intervening elements may be present.

Terms such as the “same,” as used herein when referring to layout, location, shapes, sizes, compositions, amounts, or other measures do not necessarily mean an exactly identical orientation, layout, location, shape, size, composition, amount, or other measure, and are intended to encompass nearly identical shapes, sizes, compositions, amounts, or other measures within typical variations that may occur resulting from conventional manufacturing processes.

Spatially relative terms, such as “upper,” “above,” “top,” “lower,” “bottom,” “front,” “back,” and the like, may be used herein for ease of description to describe positional relationships, such as illustrated in the figures, for example. It will be understood that the spatially relative terms encompass different orientations of the device in addition to the orientation depicted in the figures.

Ordinal numbers such as “first,” “second,” “third,” “primary,” “secondary” etc. may be used simply as labels of certain elements, steps, etc., to distinguish such elements, steps, etc. from one another. Terms that are not described using “first,” “second,” etc., in the specification, may still be referred to as “first” or “second” in a claim. In addition, a term that is referenced with a particular ordinal number (e.g., “first” in a particular claim) may be referenced elsewhere without an ordinal number or with a different ordinal number (e.g., “second” in the specification or another claim).

Any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

The following detailed description refers to the accompanying drawings. Wherever possible, the same reference numbers are used in the drawings and the following description to refer to the same or similar elements. While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure.

The current application is directed to devices, systems and methods for a top-loaded rod connector, which may be fixed or poly-axial, and which may be utilized as an adjunct to existing open and percutaneous (minimally invasive surgery - MIS) spinal rod fixation constructs, or as a new construct with new open and percutaneous spinal rod fixation constructs. The present devices and systems may be used for example in minimally invasive (MIS) or open surgery.

In alternative embodiments throughout this application, instead of a traditional rod with a circular cross-section, a different configuration rod may be used as well, including for example, those having a cross-section that is semicircular, oblong, oval, rectangular, square, trapezoidal, chamfered, etc. Thus, the term “rod” as used herein, is intended to encompass devices of congruent or non-congruent diameters. Also included are devices not only having a circular cross-section, but also devices having other shaped cross-sections (semicircular, oval, rectangular, square, trapezoidal, chamfered, etc), pre-bent or straight, and other devices that may be elongated, such as plates, beams, rails, I-beams, etc. Reference to a “rod” or “plate” or “rail” is intended to include one or any of these configurations or other suitable connection device that may be known to those skilled in the art. Additionally, the term “primary rod”, “fixation rod” and “primary fixation rod” are used interchangeably herein. The terms “secondary rod”, “connecting rod” and “secondary connecting rod” are used interchangeably herein and may also refer to any of a “rod” or “plate” or “rail”, or other suitable connection device that may be known to those skilled in the art.

Devices or systems provided herein (also referred to as “connectors”) which may include a main body and a tulip, and optionally a cannula and/or other components) may join spinal rods of congruent or non-congruent diameters. They can be used for example in revision surgery, e.g. where a previous spinal fusion surgery has failed or complications arise, to connect new rods to existing hardware, which may minimize the extent of the revision surgery. The present connectors may also be employed in complex spinal reconstructions for adult deformities, for example, when using multiple rod constructs.

The present application describes devices, systems, kits and methods for an open and percutaneous top loaded fixed cross-link, diagonal, or domino connector as an adjunct to existing open and percutaneous spinal rod fixation constructs, which may be utilized for example, as an adjunct to existing open and percutaneous spinal rod fixation constructs. Devices and methods of the present application, include a retainer-portal assembly configured to attach on to an existing rod construct, after which a rod or plate can be installed into the slot to connect or optionally cross-connect existing or additional rods or rod constructs to the original construct. The rods may then be kept in place using a retention cap. The present devices, systems and methods may or may not require any additional incisions by a surgeon, apart from those already created.

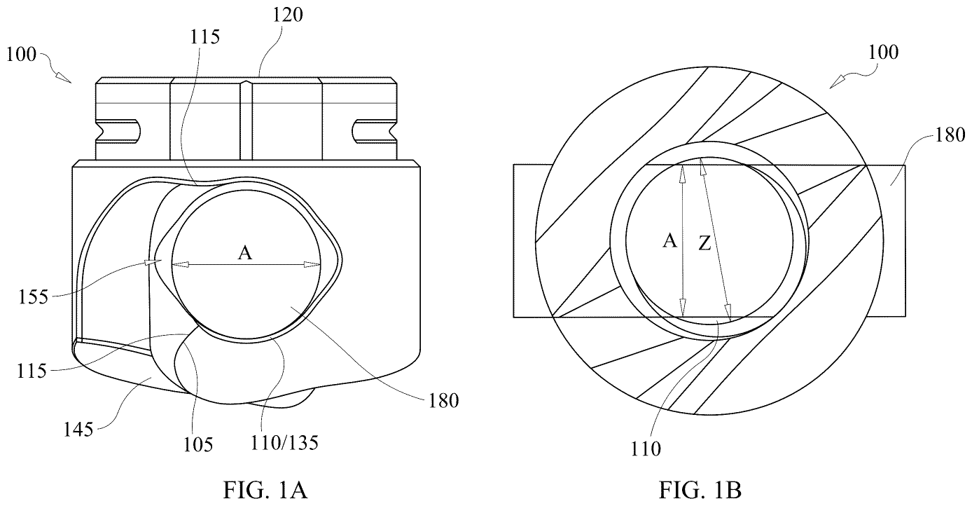

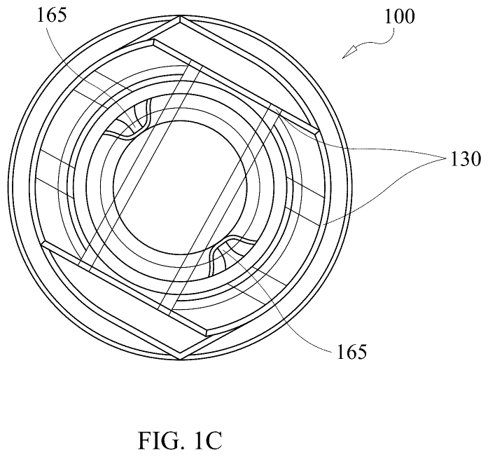

The present application provides a main body device 100. FIGS. 1A, 1B, and 1C depict a main body device 100 in accordance with example embodiments, from side, bottom, and top views, respectively. The main body 100 is configured to define an opening 110 of the main body 100. The main body 110 is configured such that the opening 110 is of a size and shape to receive and/or retain a primary rod 180 (or plate or rail, etc). The terms first rod, first primary rod, primary rod, and first primary fixation rod are used interchangeably herein and are also intended to encompass a first or primary plate or rail, throughout the present application.

In example embodiments, the present application includes devices, systems and methods that include a main body 100 and use the main body 100 to attach onto an existing rod construct for example, by attaching a primary rod 180 from an existing construct to the main body 100. The present devices and systems may further include a tulip 200 and a cannula 300. A secondary rod 280 (or plate for example) can be installed through the cannula 300 into a slot 230 in the tulip 200 defined by the tulip 200 to connect an existing or additional secondary rod 280 to the construct. The slot 230 in the tulip 200 may also be referred to herein as a “tulip slot”, “portal”, “tulip cutout” and the like.

The present devices provide a main body 100 (i.e. a rod attachment feature) which may include for example a start position opening/undersized gap 145 (defined by walls of the main body 100) of the main opening 110 configured to receive a primary rod 180 during installation of the primary rod 180 within the main body 100. The main body 100 may be configured to hook or clip or otherwise attach onto a primary rod 180. The main body 100 defines a primary rod attachment opening 135 (or first side opening), which may be for example a circle or generally circular. While a circular cutout 135 (or generally circular) is shown as the rod attachment opening 135 (or first side opening), the cutout may be circular, elliptical, rectangular, polygonal, triangular, or a combination of these shapes. The cutout 135 (or first side opening) may include at least one or two relaxation slots 155 contiguous therewith. It is noted that the primary rod 180 is not part of the present device itself. However one or more primary, secondary or further rods and/or plates may be included in systems or kits provided herein.

The present devices, which may include a main body 100, may also include one or more retention features configured to hold the primary rod 180 within and/or at a desired position of the main body 100. The retention feature(s) may include for example, inside walls of a main body defining an opening 110 or cutout of the main body 100.

A first non-limiting example a retention feature may include the main body 100 defining an undersized opening (relative to the diameter of the primary rod), such as an undersized opening, having a width “X” or “Y” relative to the diameter “Z” of the main body 100 or relative to the diameter A of the primary rod 180, which serves to provide temporary resistance during the installation of the primary rod 180 into the main body 100, for retention of the primary rod 180. Insertion of the primary rod 180 within the first retention feature 105 may result in an initial click that may be heard and/or felt by a surgeon inserting the main body 100 over a primary rod. The undersized opening may serve to provide temporary resistance and tactile feedback during the initial installation of the main body 100. The primary rod 180 or primary fixation rod may enter the main body 100 through an undersized gap 145 (having a width “X” in a first direction) in the main body 100, which may be utilized for initial rod retention.

A second non-limiting example of a retention feature may produce a second or retaining click as the primary rod is positioned within the main body. This retention feature may create a primary retention feature of the present device, for example, as the primary rod 180 is further inserted into the main body 100. The opening “Y” of the retention feature may also be undersized relative to the diameter “A” of the primary rod 180. The primary retention feature along with the secondary rod, provide resistance against the primary rod 180 rotating out of the main body 100. The second retention component or feature may include for example, one or more lips 115 for retention of the primary fixation rod 180 within the main body 100.

A cutout or opening 135 in the main body 100 (which need not be circular) on this retention component formed by the openings in the main body 100, may include lip(s) 115 for additional retention on the primary fixation rod 180. The lip(s) 115 may serve to provisionally retain the primary rod 180 at a desired position within the main body 100. According to example embodiments, the lip(s) 115 may be configured such that they provide an audible and/or palpable confirmation to a surgeon of primary rod engagement at a desired location. Example embodiments may optionally include a relaxation slot 155 of opening 110 at the end of the circular cut out 135 of opening 110, such as a helical cut out, defined by a retention feature 115 of the main body 100, to provide the ability for some elastic opening of the main body 100 to facilitate the capture of the primary rod 180 in the main body 100.

These retention features both lead to the primary rod attachment 180 within the main body 100.

In example embodiments, the main body 100 may define an opening 110, which may have helical and/or revolved cutout(s) 135 that may be circular or other shape in the main body 100. The opening 110 may act as a female mating construct to accommodate the primary rod 180. This provides a direct radial and/or axial clamping force on the primary rod 180, for retention and additional stability upon the application of axial and/or torsional loads. A width “Z” of the circular cutout 135 may be slightly larger than the diameter “A” of the primary rod, but upon compression by the tulip 200, the width is reduced to “T” which provides resistance to axial forces applied perpendicular to the primary rod. As indicated above, while a circular cutout 135 is shown, the cutout may be circular, elliptical, rectangular, polygonal, triangular or a combination of these shapes. The cutout may also utilize multiple sized shapes to be adaptable to various rod sizes.

The primary or fixation rod 180 or plate or rail may then be kept in place, for example, using a locking cap (not shown) and/or by pressure applied by the insertion of the tulip 200 into the top of the main body 100 and applying downward forces to the primary rod 180.

A top portion of the helical cut out or opening 110 may have a width “Y” at a position within the main body 100. Width “T” is between the seat of the clamp/main body 100 and the bottom of the tulip 200. The width “T” cause by compression of the primary rod 180 using the tulip 200 may be for the final rigid retention of the rod primary 180. There may be an undersized opening for example ‘Y’ between lips 115, relative to the diameter “A” of the primary rod, for example at initial and final positions of the primary rod. This width of the undersized opening between lip(s) 115 may result in the second click as a primary rod is progressed through the main body. A bottom portion of the helical cut out or opening 110 may have a width “X” between portions of the main body 100 that define the opening 110 that may also be an undersized opening with respect to diameter “A” of the primary rod. These serve to provide tactile feedback and temporary resistance to removal during installation due to axial/compressive loads to the primary rod.

In example embodiments, the present devices and systems may provide retention for the primary rod 180 by seating the primary rod 180 after for example, a second temporary retention click and then locking it in place for example, with a locking mechanism, such as a set screw or tulip 200, which may apply axial compression on the primary rod 180 perpendicular to the axis of the primary rod 180. The locking mechanism or tulip 200 may be inserted into a top of a clamp component or main body 100 and tightened such that it compresses the primary rod 180 onto the seat of the clamp, providing resistance against the primary rod 180. In example embodiments, the primary rod 180 may be secured or locked into a desired position prior to insertion of a tulip 200 into a top opening 120 of the main body 100.

According to example embodiments, widths “X” or “T” may be, or may not be undersized relative to width “Z” of the first side opening 110 and another retention feature such as the lip(s) 115 may retain a primary rod or plate 180 within the main body 100.

The main body 100 opening 110 may be circular, elliptical, rectangular, polygonal, triangular or a combination of these shapes to be suitable for receiving a primary rod 180 or plate or rail of varying shape. While a single circular opening 135 (first side opening) of opening 110 as shown in FIG. 1A for a rod 180 having a circular cross-section, multiple cut outs or opening portions may be used to accommodate varying shapes and sizes of primary fixation rods. The first side opening 135 of opening 110 may be circular, elliptical, rectangular, polygonal, triangular or a combination of these shapes. The cut out 110 may also utilize multiple sized shapes to be adaptable to various rod/rail/plate sizes.

FIG. 1B shows a bottom view of the main body of FIG. 1A according to non-limiting example embodiments. Although FIGS. 1A and 1B show a primary rod 180 through a first side opening 135 portion of the opening 110, the primary rod 180 is not part of the main body itself. The top of the main body 100 may define a top opening 120. The top opening 120 may be configured to receive a tulip 200, tool, or other accessory that may be used in the present systems and/or methods. In example embodiments, the top opening 120 may be configured to assist in positioning (such as snapping and/or rotating) and/or locking a tulip 200 to a desired location.

FIG. 1C depicts a top view of a main body 100 according to example embodiments.

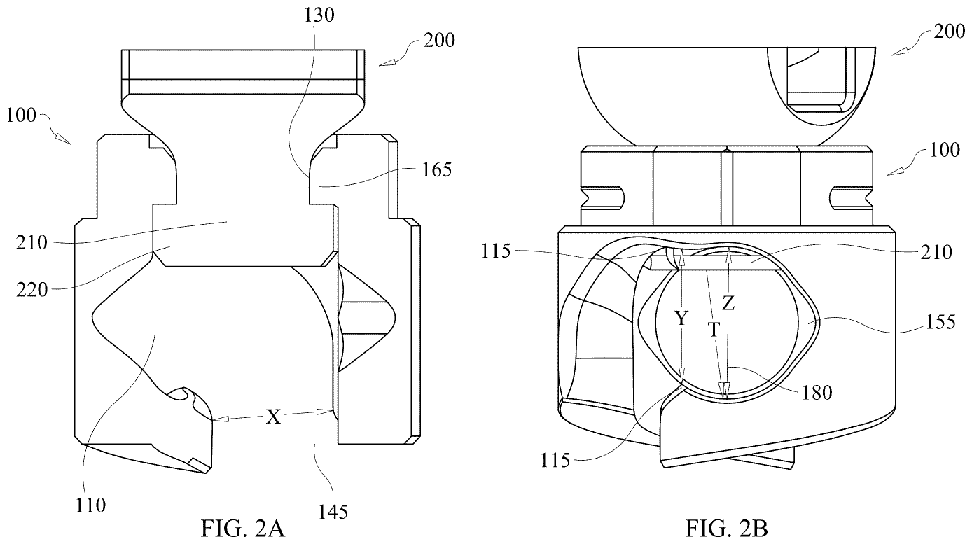

FIGS. 2A and 2B depict further embodiments of a main body 100 having a bottom portion of a tulip 200 engaged therewith. FIG. 2A is a cross-sectional view of the main body 100 with a bottom portion 210 of a tulip 200 engaged therein.

Inside walls 130 of a top portion of the main body 100 defining a top opening 120, may include a snap attachment mechanism, or a screw threading or other mechanism for accepting and/or attaching a tulip 200 thereto.

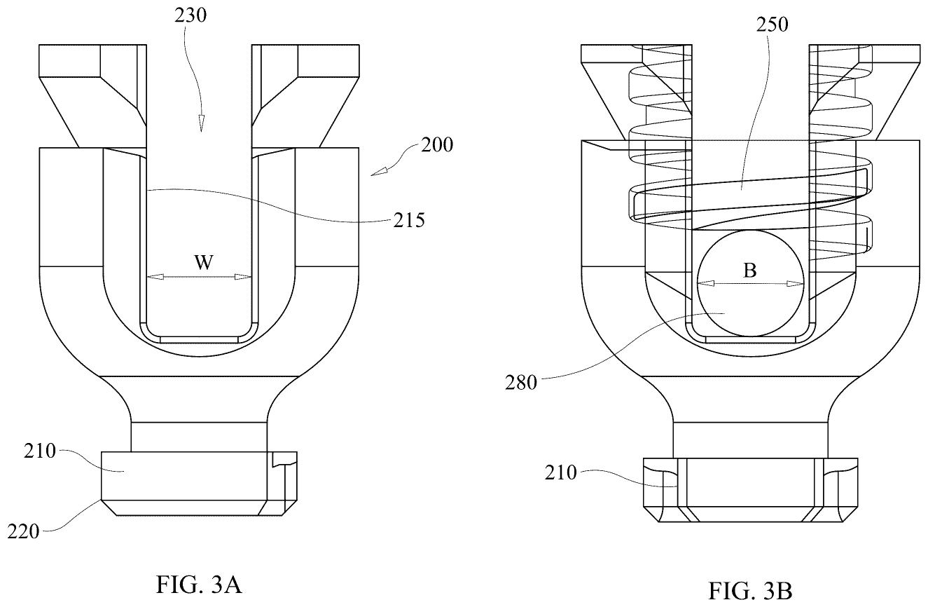

FIGS. 3A, 3B and 3C depict a tulip 200 in accordance with non-limiting example embodiments. The tulip 200 may be configured for secondary rod 280 retention in the present devices and systems. FIGS. 3A, 3B and 3C show representations of a tulip 200 defining a slot or portal 230 that is adapted to receive a connecting rod, plate or rail. The slot or portal 230 seats the secondary rod or plate or rail 280. In examples, the slot 230 shown in the Figures, such as FIG. 3B, may be larger than the prescribed secondary rod 280 diameter or widest width “w” according to example embodiments, to allow for varying sizes as well as trajectories/variations of angulation of the connecting rod or plate or rail about the anterior-posterior axis upon implantation.

Example tulips 200 may include external shelves 220 at a bottom portion 210 of the tulip 200. The tulip component 200 may insert into a top of the main body 100 clamp component 165 by passing through a cut out. The external shelves 220 on the tulip 200 component initially insert axially/vertically into the internal gap of the clamp component 165.

After the tulip 200 advances through the top opening 120 (as shown in the cross-sectional view of FIG. 2A), it rotates, compressing axially against the top surface of the primary rod 180, as can be seen in FIG. 2B, and pushes it downward onto the lower portion of the main body 100 arms. The compression is maintained and the tulip 200 is retained by an internal shelf of the clamp component 165. This provides a direct radial and/or axial clamping force on the primary fixation rod for retention and additional stability upon the application of axial and/or torsional loads. Upon compression, the relative angular position of the tulip 200 and retaining clamp may be adjusted as necessary by rotating the tulip along the internal shelf of the clamp. The tulip 200 can be rotated until the opposite retaining gap is reached on the retaining clamp.

If attached to a primary rod 180 having a circular cross-section, a secondary rod 280 may be utilized, in conjunction with a portal or slot 230 of a tulip 200 to provide an axis of rotation for direct customizable orientation of the slot 230 and indirect orientation of the connecting secondary rod, rail, or plate 280.

The tulip 200 having a slot 230 may be rigidly or semi-rigidly combined with a rod attachment feature of the main body. When fully tightened, as shown for example, in FIG. 2B, a bottom portion 210 of the tulip 200, such as a bottom of external shelves 220 of the tulip 200 contact the primary fixation rod 180 and apply compression on the primary rod 180, which locks both the position of the main body 100 (or retaining clamp) on the primary rod 180 and the angle of the slot 230 of the tulip 200.

In example embodiments, internal walls 215 of the tulip 200 that define the slot or cutout 230 may include a threaded feature that is configured to interface with a threaded retention cap 250 for radial and/or axial clamping force unto the connecting secondary rod, plate or rail 280, for retention, upon the application of axial and/or torsional loads, through its driving feature(s).

FIG. 2 depicts a slot 230 defined by internal walls 215 of the tulip 200, which slot 230 may be configured in size and shape to be utilized as a seat for the secondary rod or plate or rail 280. The slot 230 may be configured to receive a secondary rod or plate or rail 280. An example slot 230, may have a width “W” that may be larger than a width or diameter “B” of the secondary rod or plate or rail 280, to allow for varying trajectories/variations of angulation of the secondary rod or plate 280 (such as a connecting rod or cross-connecting rod or plate) about the anterior - posterior principal/vertical axis of the tulip 200 upon implantation of the present device into a patient.

While a rectangular slot 230 cutout profile is shown in FIG. 3A, according to other non-limiting embodiments, the slot 230 side profile may be circular, elliptical, rectangular, polygonal, triangular or a combination of these shapes. The cutout(s) may also be one or more following the same and/or different trajectories. One or more additional slots or openings (not shown) may be included following the same and/or different trajectories as slot 230, which are configured to receive an additional rod, plate or tool for use with the present devices, systems or methods.

The tulip 200 may have an internal threaded feature (not shown), on internal walls 215 of the tulip 200. The threaded feature may be configured to interface with an optional locking or retention cap 250 (see FIG. 3B), such as a threaded locking cap or screw. In example embodiments, locking caps provided herein may not necessarily be threaded caps. The locking cap 250 may be configured to be able to apply a radial and/or axial clamping force unto the secondary rod or plate or rail 280, for retention of the secondary rod or plate or rail 280, upon an application of axial and/or torsional loads, through its driving feature(s). The driving features of the locking cap 250 may be a portion of the locking cap 250 that a driver attaches to. The locking cap 250 can be designed to use different types of drivers (such as a screwdriver, which may be a cross, hexagon, etc).

In example embodiments, an internal threaded feature of the tulip 200 may be a female threaded feature and the locking cap 250 may be a threaded locking cap having a male threaded feature configured in size and shape to engage with a female threaded feature of the internal threaded feature of the tulip 200. Although threaded configurations for attachment of the locking cap 250 to the tulip 200, it is contemplated that a locking cap 250 may be attached to the tulip 200 by another configuration or method.

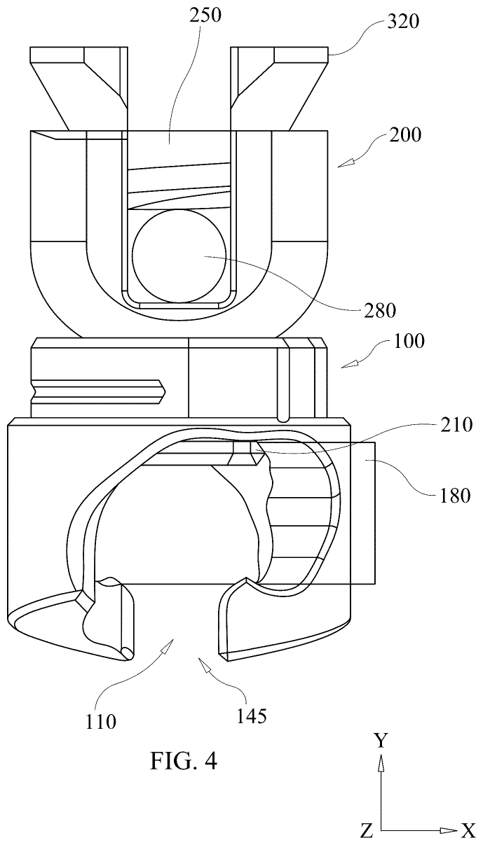

As shown for example, in FIGS. 3A, 3B, and 4, in non-limiting examples, the tulip 200 may be attached to one or more petals 320 that expand outward at the top of the tulip 200. The geometry of the tulip 200 and/or the petals 320 can be configured to facilitate alignment and engagement with corresponding device components, such as a cannula 300, for guiding the device components into a correct position for a secure connection. In example embodiments, internal walls of the top petals 320 may include a female threaded feature or other locking mechanism, to receive and/or lock at least a portion of the locking cap 250. In example embodiments, a bottom portion of the locking cap 250 may engages with inside walls of a tulip 200, while a top portion of the locking cap may engage with inside walls of the petals 320.

A function of the tulip 200 can be to provide guided engagement with mating components. The optional outwardly expanding petals 320 may be configured and shaped to guide the insertion of corresponding components, reducing the potential for misalignment and ensuring a precise connection. The tulip 200 design may provide self-aligning capabilities. As a mating component is introduced to a connector device of example embodiments, the petals 320 may flex to accommodate and guide the component into the optimal position for engagement, effectively compensating for minor alignment errors during the connection process.

Although petals 320 are depicted in in the depicted embodiments, it should be understood that petals 320 may be present, or not present, in example embodiments. For example, embodiments without petals 320 may be a short tulip configuration, which may be suitable for example for open surgery, in which tulips are not necessary. In other embodiments, for example in MIS methods, it may be useful or preferable that one or more petals 320 may be present.

The main body 100 may be configured such that it can attach to a primary rod, plate, rail etc. 180 from various manufacturers, such as rods, plates or rails that may already be in a patient. The tulip 200 may be configured such that existing rods may be used as a secondary rod.

As shown for example in FIG. 4, devices and systems provided herein may include a main body 100 and a tulip 200 connected to the main body 100. The tulip 200 and the main body 100 may be connected to one another, permanently, or in a detachable manner, for example, by use of the tulip clamping or clicking into the main body. The main body 100 and the tulip 200 may be formed of the same or different materials.

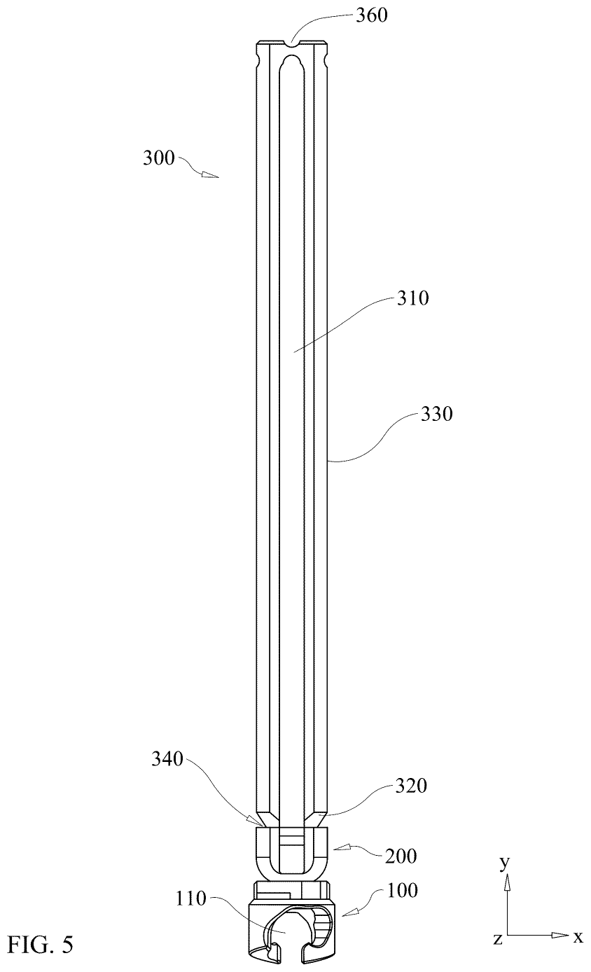

FIG. 5 depicts a representation of a working channel or cannula, created by two or more detachable tabs 330 extending from the slot 230 previously described. In example embodiments, the gap 310 between the tabs 330 is designed as a guide for the installation of the connecting (secondary) rod 280. The tabs 330 shown may be rigidly connected to create a temporary unthreaded sleeve for installation, but can be cut when necessary. Notches 340 may be implemented at the cannula-tulip junction to facilitate the detachment of the cannula/working channel from the tulip, upon application of bending or torsional loads.

While a single bodied, straight connecting rod 280, with a circular cross-section is shown in the figures, the connecting component may also be a plate, may be multi-bodied, curved, entailing circular, elliptical, rectangular, polygonal, triangular or a combination of the shapes in its cross-section.

The main body 100 and tulip 200 may also be configured to receive rods, plates or rails having the same or different sizes, such as cross-sectional diameters. In example embodiments, the tulip 200 and/or main body 100 may be rotatable with respect to one another, such that the respective openings adapted to receive a primary rod (or first plate) 180 and a secondary rod (or second plate) 280 are at a desired rotational angle with respect to one another before or after insertion of one or both of the primary and secondary rods and/or plates 180 and 280. For example, rotation of the main body 100 and tulip 200 with respect to each other may occur before or after insertion of the present system into a patient. A mechanism or lock may be provided for fixing or locking the main body 100 and tulip 200 with respect to each other at a desired angle after a desired angle is achieved. In some embodiments, the main body and/or the tulip 200 may include a locking mechanism, such as a bi-stable latch, a twist-lock system, or other lock, allowing for secure engagement and easy release of connected components. In example embodiments, the tulip 200 may interface with the top opening 120 of the main body 100, such that at least a portion of the slot 230 of the tulip 200 is directly over the opening 110 of the main body in the second direction. In example embodiments, a portion of the primary rod/post/rail 180 that is within the main body 180 is directly under (in the second direction) a portion of the secondary rod/post/rail 280 that is within the tulip 200. By specifying that an element or opening is “directly over” or “directly under” another element, this is intended to mean that the elements are at least partially (or fully) over or under one another in a direction, such as the the Y direction, as can be seen for example in FIG. 4. Thus, the elements or openings partially or fully overlap one another in another direction, such as the X direction. As used herein the term “directly over” or “directly under” does not mean that the elements directly touch or abut one another or that openings are contiguous with one another.

In alternative embodiments, the desired angle between the side opening 135 (or first side opening) of the main body 100 (or first side opening) and the slot 230 of the tulip 200 may be preset or fixed prior to insertion into the patient.

Example embodiments may include a third (or more) rod/plate/rail etc. over the locking cap of the second rod/plate/rail etc., and then add a second locking rod or plate on top of the third (or more) rod, plate, beam, rail, etc. Thus, example devices and systems may provide a mechanism or system to stack three or more rods, plates, beams, rails, etc or a combination thereof. In example embodiments, the portion of the third or more rod/plate/rail that is within the present tulip 200 or cannula 300 at least partially (or optionally fully) overlaps with the portion of the second rod/plate/rail within the tulip 200 or cannula 300 and at least partially overlaps (or optionally fully overlaps) with the portion of the first rod/plate/rail within the main body 100, in the second direction.

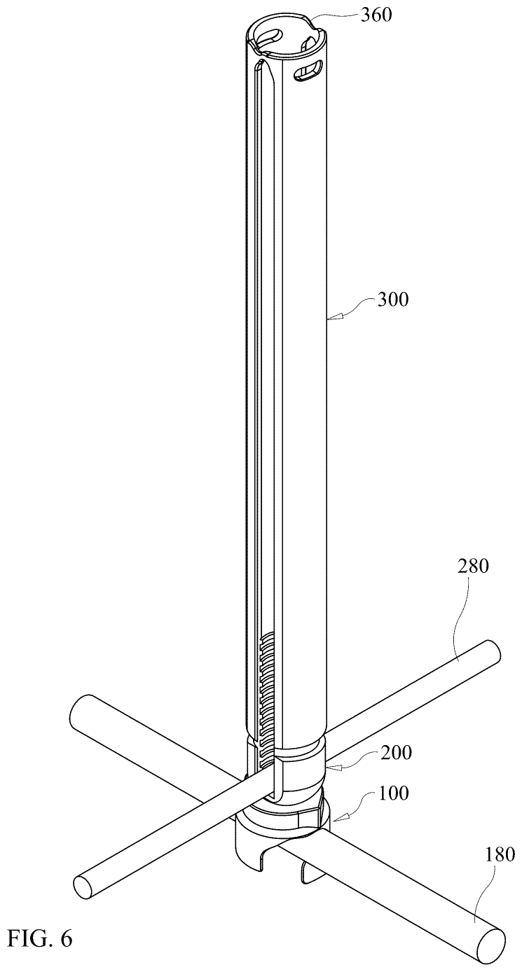

FIGS. 5 and 6 depict examples of an assembled system including a main body 100, configured to receive a primary rod, rail or plate 180, and a tulip 200 configured to receive a secondary rod, rail or plate 280, and a cannula 300 formed by two or more detachable tabs 330. A gap or opening 310 in the cannula 300 between the tabs 330 of the cannula 300 may extend from the slot 230 created by the tulip 200, such that the gap 310 and slot 230 are a contiguous opening. An opening 310 between the tabs 330 may be configured as a guide for the installation of the connecting rod or secondary rod 280.

In example embodiments, the tabs 330 may be rigidly connected to the tulip 200 to create a temporary unthreaded or partially-threaded sleeve for installation of a secondary 280 or further rod into a patient. The tabs can be cut or snipped off when necessary, or may not be rigidly connected.

In other embodiments, the tabs may not be connected at the top section 360, or may be minimally connected at the top section 360 of the cannula 300. One or more notches 340—either continuous or discontinuous—may be present at the cannula 300—tulip 200 junction, to enable or facilitate the detachment or separation of the cannula/working channel tabs from the tulip 200, upon application of bending or torsional loads by a user of the present systems (such as a surgeon). While a single-bodied, straight connecting rod, with a circular cross-section is shown, the connecting component may also be a plate or a rail and may be multi-bodied, curved, entailing circular, elliptical, rectangular, polygonal, triangular, or a combination of the shapes in its cross-section. Multiple angles that can be accomplished between the primary and secondary rods, for example, based on the orientation of the tulip 200 with respect to the main body 100. The tulip 200 may be inserted in the main body at a desired orientation 100. Or in other example embodiments, the tulip 200 may be inserted in the main body 100 and then adjusted in position after insertion, to achieve a desired position with respect to the main body 100.

FIG. 5 depicts a representation of a working channel or cannula 300, created by two or more tabs 330 extending from the tulip 200 described herein having a gap or opening 310 in the cannula 300. In the depicted example, the side opening 135 of the main body is aligned over the slot 230 of the tulip in the second direction. In the depicted example, the side opening 135 and the slot 230 face the same direction so rods inserted therein would be parallel with respect to one another.

In FIGS. 5 and 6, the tabs 330 may be detachable from the tulip 200 and/or from each other. An opening or gap 310 between and defined by the tabs 330, is configured as a guide for the installation of the connecting secondary rod 280.

As shown for example in FIG. 5, systems provided herein may include a main body 100 and a tulip 200 connected to the main body 100, and further a cannula 300 connected to the tulip 200. In example embodiments the cannula 300 may be connected to the tulip 200 prior to insertion into a patient, or it may be connected to the tulip 200 after insertion into a patient.

In example embodiments, the opening 135 (which is the cutout side opening of opening 110) of the main body 100 and opening or slot 230 of the tulip 200 are aligned over one another in the second direction, such that when primary rod 180 and secondary rod 280 are inserted in the side opening 135 (or first side opening) and the tulip slot 230, the primary and secondary rods 180 and 280 overlap with one another at least partially in the Y direction, regardless of the rotation of tulip 200 with respect to the main body 100.

FIG. 6 depicts a rotated configuration of devices and systems including a main body 100, tulip 200 and cannula 300, exhibiting a different angle of the tulip with respect to the main body 100, and shows that by including (or rotating) the tulip 200 and main body 100 with respect to one another, perpendicular and other oblique angles can be accomplished between the primary rod 180 and the secondary rod 280, for example in embodiments where the tulip 200 is aligned with or rotated to a rotated position with respect to the main body 100. In example embodiments, the position of the primary rod 180 with respect to the secondary rod (as viewed from a top view) depends on the relative angular position of the slot 230 and side opening 135 of the main body 100 (or first side opening). In example embodiments, the tulip 200 is over the main body 100 and aligned vertically, such that their respective openings in which secondary 280 and primary rods 180 may be inserted, are also over one another or “inline” with one another.

In example embodiments, the primary and secondary rods 180 and 280, respectively overlap with one another, for example, with the secondary rod 280 over the primary rod 180. The rods may be at any angle with respect to one another - in that they may be parallel with respect to one another or they may be at oblique angles with respect to one another.

FIG. 6 depicts a configuration of the present system including a main body 100, tulip 200 and cannula 300, in which the primary rod 180 and the secondary rod 280 are in a cross connector configuration. In particular, a portion of the primary rod 180 within the main body overlaps in a second direction with a portion of the secondary rod 280 within the tulip 200. In example embodiments, the primary rod 180 and the secondary rod 280 may be rotated with respect to one another such that they are perpendicular with respect to one another.

Components of the present devices may be made of various materials. According to example embodiments, components such as the main body 100, the tulip and/or a cannula may be a rigid or semi-rigid material for stability and/or to hold their shapes. For example, the tulip 200 and the main body 100 may be made of pure titanium or titanium alloys (for instance, titanium-aluminum-molybdenum alloy) thereof, stainless steel, Cobalt-Chromium, molybdenum rhenium or other materials known by those skilled in the art to be suitable implants. The various locking mechanisms, caps, rods, and other components described herein may also be of various same or different suitable materials.

Kits provided herein may include one or more of the system components described herein. For example, kits may include one or more of the following: a main body, a tulip, a cannula or component thereof, instructions for use of the present devices and systems, one or more rods or plates, such as the primary or secondary rods or plates, locking mechanisms and/or caps for use with the present devices and systems, or tools for assembly or insertion of the present devices or systems, or components thereof.

Also provided herein are methods of connecting spinal rods using the devices and systems provided herein. Non-limiting examples of the present methods may include the following, attaching the main body 100 unto an existing primary rod 180, for example a rod already within a patient, attaching the tulip 200 onto the main body 100, for example by screwing the tulip 200 into the main body 100, placing a secondary rod 280 into the tulip 200, adjusting the tulip 200 and/or secondary rod 280 to a desired angle with respect to the main body 100 and/or primary rod 180, and driving a locking cap 250 into the tulip 200. In example embodiments, the locking cap 250 may be provided to the desired location through a cannula 300 including tabs 330 to compress/fixate the secondary rod 280. The methods may further include detaching tabs 330 from each other and from the tulip 200.

According to examples, methods may include attaching an end of the secondary rod 280 to a pedicle screw or any other device designed to work with spinal rods. The present methods may be minimally invasive methods that include utilization of a tulip with detachable tabs for visibility outside the patient's body and the placement of the secondary rod through the slot. Alternatively, methods may be provided for using the present systems and devices that are open methods.

By way of non-limiting example, the present methods may be used in MIS or open surgery methods on the spine, in all spine regions. For example, the present methods and devices may be used with respect to the thoracolumbar spine, or for cervical or sacral regions, but are not limited thereto.

Methods of using the present systems may or may not require additional incisions into the patient, apart from those already created. For example, the present methods may or may not require incisions in addition to prior incisions from a prior surgery, or from a prior/different procedure, within the same surgery.

The present devices systems and methods provide for example, top-loading of the main body 100, which allows a surgeon to place the device in an MIS or open surgery.

The present devices systems and methods also provide an ability for the tulip 200 to be placed percutaneous simultaneously with the main body 100, or after the main body 100 has been placed within a patient. The tulip 200 may also be polyaxial with respect to the main body 100, for example, by attaching (such as clicking or clamping) the tulip 200 into the main body 100, and adjusting the tulip 200 to a different polyaxial position within the main body 100, or they may be fixed in position with respect to each other prior to insertion. A locking mechanism, such as a screw or other device may be used to fix the position of the tulip with respect to the position of the main body 100.

The present devices, systems and methods also provide an ability for percutaneous (MIS) or open surgery placement and retention of a secondary rod 280, due to the characteristics of the tulip 200, such as slots, threading for the locking or retention cap 250, which may be configured to retain/compress the secondary rod 280, and optional extended tabs/petals 270, which can be separated from each other and/or broken off.

The present devices, systems, kits and methods provide a significant advancement in the field of minimally invasive spine surgery, addressing limitations of previous devices and methods, and potentially improving patient outcomes through enhanced construct stability and reduced surgical invasiveness.

While the present disclosure has been described in terms of exemplary aspects, those skilled in the art will recognize that the present disclosure can be practiced with modifications and/or variations within the spirit and scope of the application. The examples provided herein are merely illustrative and are not meant to be an exhaustive list of all possible configurations, aspects, applications or modifications of the present disclosure.

What is claimed is:

1. A connector system comprising:
a main body defining a main body opening configured for insertion of a primary rod, rail or plate therein,
a tulip defining a tulip opening configured for insertion of a secondary rod, rail or plate therein, the tulip being inserted into a top opening in the main body and attached to the main body above the main body, and
a retention cap configured to maintain the secondary rod, rail or plate at a desired position therein within the tulip;
wherein the main body and the tulip are aligned in a first direction such that the main body opening and the tulip opening are directly over each other in a second direction that intersects the first direction, thereby allowing a portion of the primary rod, rail or plate and a portion of the secondary rod, rail or plate to be alignable directly over one another in the second direction.

2. The connector system of claim 1, further comprising a cannula detachably attached to the tulip.

3. The connector system of claim 2, wherein the cannula comprises at least two tabs defining a channel therebetween.

4. The connector system of claim 2, wherein the cannula is aligned directly over the tulip and the main body.

5. The connector system of claim 1, wherein the main body further includes at least one lip configured to retain the primary rod, rail or plate.

6. The connector system of claim 5, wherein the at least one lip is configured to provide an audible or palpable confirmation of primary rod, rail or plate engagement to a desired location within the main body.

7. The connector system of claim 1, wherein the tulip includes internal helical threads and the retention cap includes corresponding helical threads such that the retention cap is threadable in the tulip.

8. The connector system of claim 1, wherein the tulip is rotatable in a first direction with respect to the main body so the main body opening is at a desired angle with respect to the tulip opening.

9. The connector system of claim 1, wherein the main body and the tulip are rotatable with respect to one another such that the secondary rod, rail or plate in the tulip is parallel, perpendicular or at another angle with respect to the primary rod, rail or plate in the main body.

10. The connector system of claim 1, wherein a top loading tulip is configured to retain the primary rod, rail or plate at a desired position in the main body.

11. The connector system of claim 1, further comprising at least one petal.

12. A kit including the main body and the tulip of claim 1.

13. The kit of claim 12, further comprising a cannula aligned over the tulip and the main body in the second direction.

14. The kit of claim 12, further comprising at least one additional component selected from the group consisting of instructions for use of the system, at least one of the primary rod, rail or plate or the secondary rod, rail or plate the locking screw, the retention cap, and a tool for assembly or insertion of the connector system or a component thereof.

15. A method of connecting multiple spinal rods, rails or plates comprising
attaching a main body to a primary rod, rail or plate,
attaching a tulip to the main body,
placing a secondary rod, rail or plate into an opening of the tulip,
adjusting the tulip or the secondary rod, rail or plate, such that the secondary rod, rail or plate has a desired angle with respect to the primary rod, rail or plate, and
driving a retention cap into the tulip to lock the secondary rod, rail or plate at a desired position,
wherein a portion of the primary rod, rail or plate within the main body is directly aligned in a vertical direction with a portion of the secondary rod, rail or plate within the tulip.

16. The method of claim 15, further comprising attaching a cannula to the tulip, the cannula being configured to be suitable for inserting or guiding the retention cap to the tulip.

17. The method of claim 16, wherein the cannula comprises one or more tabs that are configured to be detachable from each other and detachable from the tulip.

18. The method of claim 17, further comprising detaching the tabs from each other and from the tulip.

19. The method of claim 15, further comprising attaching the secondary rod, rail or plate to a pedicle screw.

20. The method of claim 15, wherein the method is an open or minimally invasive method.
